SEQUENCE LISTING (3 sequences)

SEQ ID NO: 1 - protein, 175 aa, Homo sapiens
MMLPMTLCRMSWMLLSCLMFLSWVEGEESQKKLPSSRITCPQGSVAYGSYCYSLILIPQTWSNAELSCQMHFSGHLAFLLSTGEITFVSSLVKNSLTAYQYIWIGLHDPSHGTLPNGSGWKWSSSNVLTFYNWERNPSIAADRGYCAVLSQKSGFQKWRDFNCENELPYICKFKV

SEQ ID NO: 2 - protein, 20 aa, Homo sapiens
IGLHDPSHGTLPNGSGWKWS

SEQ ID NO: 3 - protein, 15 aa, Homo sapiens
IGLHDPSHGTLPNGS

(19) United States
(12) Patent Application Publication
(10) Pub. No.: US 2008/0171704 A1
(43) Pub. Date: Jul. 17, 2008

(54) Composition and method for treating diabetes

(76) Inventors: Aaron I. Vinik, Norfolk, VA (US); Lawrence Rosenberg, Montreal (CA); Gary Pittenger, Virginia Beach, VA (US); David Taylor-Fishwick, Norfolk, VA (US); Michael Salem, Ft. Lauderdale, FL (US); Scott Mohrland, Plantation, FL (US)

Correspondence Address: LOTT & FRIEDLAND, P.A., ONE EAST BROWARD BLVD., SUITE 1609, FORT LAUDERDALE, FL, 33301, US

(21) Appl. No.: 11/999,208

(22) Filed: Dec. 4, 2007

Related U.S. Application Data
(62) Division of application No. 10/253,733, filed on Sep. 24, 2002, now abandoned.
(60) Provisional application No. 60/329,330, filed on Oct. 16, 2001.

(51) Int. Cl. A61K 38/10; A61P 3/00

(52) U.S. Cl. 514/14

(57) ABSTRACT

The present invention comprises dosing regimens and formulations of islet cell neogenesis associated protein (INGAP) and INGAP Peptide. The formulation disclosed herein is shown have acceptable stability as a pharmaceutical composition. Further, the formulation is able to regenerate functional islets.

### CROSS REFERENCE TO RELATED APPLICATIONS

This application is a Divisional Application of parent application Ser. No. 10/253,733, filed on Sep. 24, 2002, which claims the benefit of provisional application Ser. No. 60/329,330 filed on Oct. 16, 2001, which are herein incorporated by reference in their entirety.

### BACKGROUND

Pancreatic islet cell mass is lost in type I diabetes mellitus, a disease in which a progressive autoimmune reaction results in the selective destruction of insulin-producing β-cells. In type 2 diabetes mellitus, so-called adult-onset disease, but also increasingly a condition in young overweight people, the β-cell mass may be reduced by as much as 60% of normal. The number of functioning β-cells in the pancreas is of critical significance for the development, course, and outcome of diabetes. In type I diabetes, there is a reduction of β-cell mass to less than 2% of normal. Even in the face of severe insulin resistance as occurs in type II diabetes, the development of diabetes only occurs if there is an inadequate compensatory increase in β-cell mass. Thus, the development of either major forms of diabetes can be regarded as a failure of adaptive β-cell growth and a subsequent deficiency in insulin secretion. The ability to stimulate the growth of islets and β-cells from precursor cells, known as islet neogenesis, would be a novel and attractive approach to the amelioration of diabetes.

Through a series of experiments a pancreatic extract, termed ilotropin, was prepared and demonstrated to stimulate β-cell neogenesis from pre-existing progenitor cells associated with the pancreatic ductal system. Based on the hypothesis that pancreatic ductal cell transformation leading to islet neogenesis is dependent upon endogenous growth factors, genes, and protein products, a search ensued to identify the active ingredient in ilotropin. This line of investigation led to the discovery of a novel gene and its associated protein, the islet neogenesis associated protein (INGAP).

INGAP Peptide (INGAP¹⁰⁴⁻¹¹⁸), a 15 amino acid sequence contained within the 175 amino acid INGAP, has been shown to stimulate ductal cell proliferation in hamsters. INGAP Peptide is amino acids 103-117 of SEQ ID. NO: 2 of U.S. Pat. No. 5,834,590 which is incorporated herein by reference.

### SUMMARY OF THE INVENTION

The present invention comprises dosing regimens and formulations of INGAP Peptide. The formulation disclosed herein is shown to have acceptable stability as a pharmaceutical agent and adequate safety for human clinical trials. INGAP Peptide thus prepared is further shown to regenerate functional islet cells that maintain normal feedback controls.

Thus, it is an object of the present invention to provide a pharmaceutically acceptable and stable composition of INGAP Peptide that is involved in islet of Langerhans neogenesis.

Another object of the invention is to provide methods for treating diabetes in a mammal.

It is another object of the invention to provide methods for treating abnormal physiological glucose regulation in a mammal.

It is another object of the invention to provide methods of increasing the number of pancreatic beta cells or islets of Langerhans in a mammal.

It is another object of the invention to provide a method of treating mammals receiving islet cell transplants.

It is another object of the invention to provide a method for inducing differentiation of pancreatic progenitor cells.

All documents cited are, in relevant part, incorporated herein by reference; the citation of any document is not to be construed as an admission that it is prior art with respect to the present invention.

### DESCRIPTION OF DRAWINGS AND FIGURES

FIG. 1. INGAP peptide treated ARIP cells (a rat pancreatic duct cell line) showing a dose dependant increase in cell number.

FIG. 2. Increase in pancreatic endocrine cell mass with INGAP Peptide. Normal Syrian golden Hamsters were treated with saline (control) or INGAP Peptide IP for either 10 days or 30 days. The number of islets/ml was determined with a computer-aided Morphometric system employing Image-Pro Plus software.

FIG. 3. Time course of blood glucose following injection of INGAP Peptide 250 mg/kg twice daily or saline for 39 days to C57BL/6J mice rendered diabetic using a regimen of multiple injections of low-dose streptozotocin. Blood glucose levels were measured every three days. Animals were followed for an additional nine days after discontinuation of INGAP Peptide injections and demonstrated the persistence of the treatment effect. * p<0.025 compared with saline-treated animals.

FIG. 4. Immunocytochemical characteristics of the pancreas of streptozotocin-diabetic C57BL/6J mice treated with INGAP Peptide. The upper left panel shows an islet still associated with a segment of duct epithelium stained with anti-insulin antibody, which demonstrates a normal presence and distribution of insulin protein. The lower left panel shows the same islet stained with a mixture of anti-glucagon and anti-somatostatin antibodies also demonstrating a normal distribution of these islet cell proteins in the islet mantle region. The upper right panel shows a newly formed islet budding off a duct stained with H & E stain. The lower right panel shows an insulin-positive cell in the wall of the duct.

FIG. 5. INGAP Peptide stimulates PDX-I expression in pancreatic ducts of C578/6J mice. Panel on the right is a second area of PDX-I immunoreactivity in association with islet cell neogenesis. Darkly stained material is the appearance of PDX-I of the cells in the duct wall.

FIG. 6. Histological characteristics of the pancreas of streptozotocin-treated C578L/6J mice. The lower right panel (STZ-INGAP) shows the pancreas of an INGAP Peptide-treated animal with an area of islet cell neogenesis, observed as cells budding from an adjacent intralobular ductule. The lower left panel (STZ-INGAP) is a normal appearing islet in the pancreas of an animal treated with INGAP Peptide. The upper right panel (STZ) shows the pancreas of a saline treated animal showing a necrotic islet with inflammatory cell infiltration characteristic of insulitis. The upper left panel (Ctrl) shows the pancreas of a normal aged-matched control mouse showing the histological appearance of a normal islet for comparison. Note that INGAP restores islet histology towards normal.

FIG. 7. Immunopositive tissue area in normal mouse following treatment with INGAP Peptide for 31 days. Data are mean ±SEM. The percentage of cross-sectional area stained for insulin was determined with an Olympus microscope (150×) and image analysis software (Image Pro Plus Version 4.0, Media Cybernetics).

FIG. 8. Effects of daily injection of INGAP Peptide to dogs for 34 days on the area of cells in the pancreas that becomes positive for insulin by immunohistochemical staining. Data are mean ±SEM. The percentage of cross-sectional area stained for insulin was determined with an Olympus microscope (150×) and image analysis software (Image Pro Plus Version 4.0, Media Cybernetics).

### DETAILED DESCRIPTION OF THE INVENTION

### Glossary of Terms

The following is a list of definitions for terms used herein.

A “Pharmaceutically-acceptable salt” is a cationic salt formed at any acidic (e.g., carboxyl) group, or an anionic salt formed at any basic (e.g., amino, alkylamino, dialkylamino, morphylino, and the like) group on the compound of the invention. Since INGAP Peptide is zwitterionic, either salt is possible and acceptable. Many such salts are known in the art. Preferred cationic salts include, but are not limited to, the alkali metal salts (such as sodium and potassium), alkaline earth metal salts (such as magnesium and calcium) and organic salts, such as ammonium. Preferred anionic salts include halides, sulfonates, carboxylates, phosphates, and the like. Clearly contemplated in such salts are addition salts that may provide an optical center, where once there was none. For example, a chiral tartrate salt may be prepared from the compounds of the invention, and this definition includes such chiral salts. Salts contemplated are nontoxic in the amounts administered to the patient-animal, mammal or human. Examples of appropriate acid-addition salts include, but are not limited to hydrochloride, hydrobromide, hydroiodide, sulfate, hydrogensulfate, acetate, trifluoroacetate, nitrate, citrate, fumarate, formate, stearate, succinate, maleate, malonate, adipate, glutarate, lactate, propionate, butyrate, tartrate, methanesulfonate, trifluoromethanesulfonate, p-toluenesulfonate, dodecyl sulfate, cyclohexanesulfamate, and the like.

“Biohydrolyzable esters” are esters of compounds of the invention, where the ester does not essentially interfere, preferably does not interfere, with the bioactivity of the compound, or where the ester is readily converted in a host to yield an active compound. Many such esters are known in the art, as described in U.S. Pat. No. 4,783,443, issued to Johnston and Mobashery on Nov. 8, 1988. Such esters include lower alkyl esters, lower acyloxy-alkyl esters (such as acetoxymethyl, acetoxyethyl, aminocarbonyloxymethyl, pivaloyloxymethyl and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters and alkylacylaminoalkyl esters (such as acetamidomethyl esters).

The term “treatment” is used herein to mean that, at a minimum, administration of a compound of the present invention mitigates a disease associated with the abnormal physiological glucose regulation in a subject, preferably in a mammalian subject, more preferably in humans. Thus, the term “treatment” includes: preventing an abnormal physiological glucose regulation mediated disorder in a subject, particularly when the subject is predisposed to acquiring the disease, but has not yet been diagnosed with the disease; inhibiting the abnormal physiological glucose regulation mediated disorder; and/or alleviating or reversing the abnormal physiological glucose regulation mediated disorder. Insofar as the methods of the present invention are directed to preventing the abnormal physiological glucose regulation mediated disorder, it is understood that the term “prevent” does not require that the disease state be completely thwarted (Webster's ninth collegiate dictionary). Rather, as used herein, the term preventing refers to the ability of the skilled artisan to identify a population that is susceptible to the abnormal physiological glucose regulation mediated disorders, such that administration of the compounds of the present invention may occur prior to onset of the abnormal physiological glucose regulation mediated disorder. The term does not imply that the disease state be completely avoided. The population that is at risk of an abnormal physiological glucose regulation mediated disorder (e.g. type I and type II diabetes), are those who have a genetic predisposition to diabetes as indicated by family history of the disease. Other risk factors include obesity or diet.

### Manufacturing and Stability

INGAP Peptide is a 15 amino acid sequence consisting of amino acids number 104-118 contained within the native 175 amino acid INGAP. INGAP Peptide can be synthesized through any of various means known in the art although the preferred means of synthesis is through 9-fluorenylmethoxycarbonyl (Fmoc) solid-phase synthesis. The preferred form of INGAP Peptide is the INGAP Peptide in a pharmaceutically acceptable salt form, preferably acetate salt. Formation of salts of peptides is known in the art. Fmoc synthesis is described in U.S. Pat. No. 4,108,846. Fmoc uses piperidine to cleave the methoxycarbonyl (moc) and trifluoroacetic acid (TFA) to cleave the peptide from the resin. INGAP manufactured according to this process can be readily purified by preparative HPLC chromatography.

INGAP Peptide has the following amino acid sequence: NH₂-Ile-Gly-Leu-His-Asp-Pro-Ser-His-Gly-Thr-Leu-Pro-Asn-Gly-Ser-COOH (SEQ ID NO: 3)

The INGAP Peptide has a chemical formula of C₆₄H₁₀₀N₂₀O₂₂, a molecular weight of 1501.6±1 Daltons and a specific rotation of −103.20 in 1% acetic acid.

The structure of INGAP Peptide is confirmed by amino acid analysis in which the INGAP Peptide molecule is hydrolyzed to its constituent amino acids. The amino acids are quantitated and shown to be present in the correct molar ratio based on the molecular structure. The molecular mass of the peptide can be determined utilizing electrospray mass spectrometry and should be in agreement with the calculated, theoretical mass of the molecule (1501.6±1 mass unit).

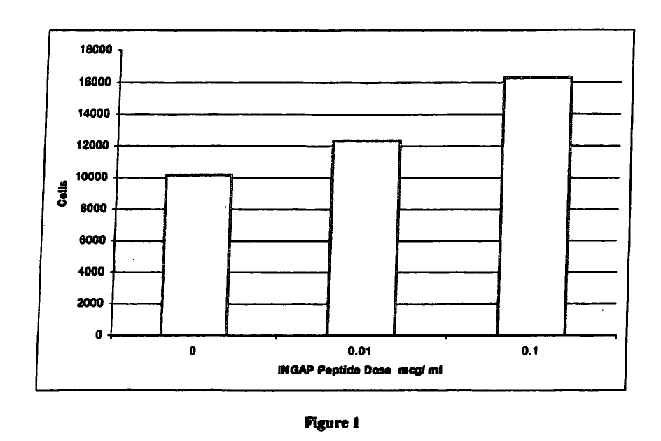

To confirm that the synthetic molecule is bioactive, a bioassay may be used to confirm the activity. ARIP cells, a rat pancreatic duct cell line, obtained from ATCC (Manassas, Va.) are used in the assay. Cells are plated into a 96-well culture plate at 10,000 cells/well, and cultured for 24 hours in Dulbecco's Minimal Essential Medium (DMEM) containing 10% fetal bovine serum. After 24 hours, the medium is replaced with DMEM without serum. Duplicate wells are treated with varying doses (0, 10⁻³ and 10⁻⁵ g/ml) of INGAP Peptide. After 21 hours, the medium is supplemented with bromodeoxyuridine (BrdU) labeling solution from a BrdU cell proliferation ELISA kit (Roche Molecular Biochemicals) and cultured for a further 3 hours. At 24 hours the cells are dried at 60° C. for 60 minutes, fixed and denatured. They are exposed to BrdU antibody for 90 minutes and developed for 15 minutes, all according to kit instructions. BrdU labeling is quantitated on a Wallac Victor 1420 Multilabel Counter. Results are compared against a standard curve of cells grown on the same culture plate, seeded at densities from 100 to 20,000 cells per well. As shown in FIG. 1, when using this assay there is approximately a 1.6-fold increase in cell count compared with controls in cultures treated with 0.1 [symbol] g/ml of INGAP Peptide.

### Stability of Bulk INGAP Peptide

Stability is determined by comparing various parameters including, but not limited to, degree of purity, total percentage of impurities, percentage of individual impurities (as determined by HPLC or other suitable quantitative method), appearance, and water content of the sample. An HPLC method can be used to determine any increase in the levels of degradation products relative to the level of INGAP Peptide. INGAP Peptide samples (both solution and lyophilized powder) are stored at various temperatures, in the presence or absence of humidity, and in light or dark vials. Degradation during different storage conditions can lead to an increase in impurities and decrease in INGAP Peptide content. It is desirable that the sample preparation is more than 80% pure, preferably more than 90% pure, more preferably more than 95%, and most preferably more than 97% pure.

The INGAP Peptide as a lyophilized powder is stable under various storage conditions. Purity of the INGAP Peptide is maintained under these conditions and degradation products are lower than the acceptable levels. Further storage up to six-months does not cause any noticeable degradation of the INGAP Peptide.

### Compositions

Another aspect of this invention is compositions which comprise: (a) a safe and effective amount of peptide of the present invention; and (b) a pharmaceutically acceptable carrier. Standard pharmaceutical formulation techniques are used, such as those disclosed in Remington's Pharmaceutical Sciences, Mack Publishing Company, Easton, Pa., most recent edition.

A “safe and effective amount” means an amount of the peptide of the invention sufficient to significantly induce a positive modification in the condition to be treated, but low enough to avoid serious side effects (such as toxicity, irritation, or allergic response) in an animal, preferably a mammal, more preferably a human subject, in need thereof, commensurate with a reasonable benefit/risk ratio when used in the manner of this invention. The specific “safe and effective amount” will, obviously, vary with such factors as the particular condition being treated, the physical condition of the subject, the duration of treatment, the nature of concurrent therapy (if any), the specific dosage form to be used, the carrier employed, the solubility of the peptide therein, and the dosage regimen desired for the composition. One skilled in the art may use the following teachings to determine a “safe and effective amount” in accordance with the present invention. Spilker B., Guide to Clinical Studies and Developing Protocols, Raven Press Books, Ltd., New York, 1984, pp. 7-13, 54-60; Spilker B., Guide to Clinical Trials, Raven Press, Ltd., New York, 1991, pp. 93-101; Craig C., and R. Stitzel, eds. Modern Pharmacology, 2d ed., Little, Brown and Co., Boston, 1986, pp. 127-33; T. Speight, ed., Avery's Drug Treatment: Principles and Practice of Clinical Pharmacology and Therapeutics, 3d ed., Williams and Wilkins, Baltimore, 1987, pp. 50-56; R. Tallarida, R. Raffa and P. McGonigle, Principles in General Pharmacology, Springer-Verlag, New York, 1988, pp. 18-20.

The peptide of the invention is dissolved or suspended in a pharmaceutically acceptable buffer. The buffer that the peptide is dissolved in can affect the pH, solubility and therefore the bioavailability of the peptide. Choice of buffer varies depending on the peptide composition, route of administration, and extent of solubility of the peptide desired, half-life of the peptide in physiological setting, and pH and buffering capacity of the physiological fluid. The pH of a favored buffer may be closer to pK, value of the peptide, or it may be dependent upon the physiological setting where the peptide is to be delivered. Suitable buffers include, but are not limited to, phosphate, acetate, carbonate, bicarbonate, glycine, citrate, imidizole and others. Particularly preferred buffer is an acetate buffer.

In addition to the subject peptide, the compositions of the subject invention contain a pharmaceutically acceptable carrier. The term “pharmaceutically-acceptable carrier,” as used herein, means one or more compatible solid or liquid filler diluents or encapsulating substances which are suitable for administration to an animal, preferably a mammal, more preferably a human. The term “compatible”, as used herein, means that the components of the composition are capable of being comingled with the subject peptide, and with each other, in a manner such that there is no interaction that would substantially reduce the pharmaceutical efficacy of the composition under ordinary use situations. Pharmaceutically-acceptable carriers must, of course, be of sufficiently high purity and sufficiently low toxicity to render them suitable for administration to the animal, preferably a mammal, more preferably a human being treated. The choice of a pharmaceutically acceptable carrier to be used in conjunction with the subject compound is basically determined by the way the peptide is to be administered. If the subject peptide is to be injected, the preferred pharmaceutically acceptable carrier is prepared sterile, with a blood-compatible colloidal suspending agent.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically acceptable carrier, in compositions for parenteral administration, comprises at least about 90% by weight of the total composition.

The compositions of this invention are preferably provided in unit dosage form. As used herein, a “unit dosage form” is a composition of this invention containing an amount of INGAP Peptide that is suitable for administration to an animal, preferably a mammal, more preferably a human subject, in a single dose, according to a good medical practice. These compositions preferably contain from about 0.1 mg (milligrams) to about 300 mg, and more preferably from about 5 mg to about 150 mg of INGAP Peptide. The frequency of treatment with the composition of the invention may be changed to achieve the desired bolus as well as to avoid side effects. Thus, no limiting examples of treatment schedules include daily, twice daily, three times daily, weekly, biweekly, monthly, and combinations thereof. Alternatively, the composition of the invention may also be administered as a continuous infusion.

The compositions of this invention may be in any of a variety of forms, suitable, for example, for oral, topical, nasal, or parenteral administration. Depending upon the particular route of administration desired a variety of pharmaceutically acceptable carriers well known in the art may be used. These include solid or liquid fillers, diluents, hydrotropes, surface-active agents, and encapsulating substances. Optional pharmaceutically active materials may be included, which do not substantially interfere with the activity of the INGAP Peptide. The amount of carrier employed in conjunction with the INGAP Peptide is sufficient to provide a practical quantity of material for administration per unit dose of the INGAP Peptide. Techniques and compositions for making dosage forms useful in the methods of this invention are described in the following references: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

### INGAP Peptide Formulation

A preferred INGAP Peptide formulation is a solution for injection using sterile water and sodium chloride as needed to adjust tonicity and produced at four different concentrations: 0, 7.5, 30, and 120 mg/0.5 ml/vial. Hydrochloric acid and sodium hydroxide may be used as necessary to adjust the pH to the preferred level. Additional concentrations may be prepared by diluting the higher concentration stocks using isotonic saline. Dilution does not affect the biological potency of INGAP Peptide.

Thus prepared INGAP Peptide formulation is stable within the pH range of 4 to 6 when stored at 5° C. and placed in either dark or light containers. However, some degradation is observed when the composition is stored at 25° C. The degradation is more evident for composition with pH of 6 than with pH of 4.5. It appears that INGAP Peptide is more stable when stored below 8° C. and below pH of 6.

### EXAMPLE 1 INGAP PEPTIDE SOLUTION FOR INJECTION

A solution of 120 mg of INGAP Peptide is prepared with the following specifications:

TABLE 1

| Parameter | Specifications |
|---|---|
| Appearance | Clear colorless solution |
| Assay | Each vial contains |
| | 90.0% to 110.0% of INGAP Peptide |
| Impurities | Each Impurity: 1.0% |
| | Total Impurities: 3.0% |
| pH | 4.0 to 5.0 |
| Bacterial Endotoxins | NMT 2.92 EU/mg |
| Sterility | Complies with USP |

### EXAMPLE 2 ADMINISTRATION OF INGAP PEPTIDE TO NORMAL HAMSTERS

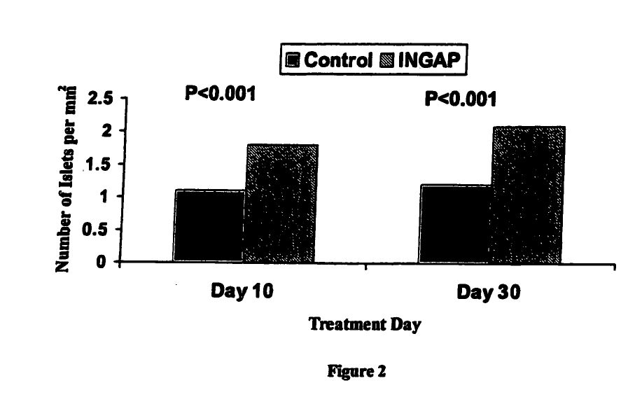

INGAP Peptide was studied for its effects on islet formation in normal hamsters. INGAP Peptide 5 mg/kg (25 mg/m²) was given IP daily for 4 weeks and β-cell mass was assessed at 10 days and at 30 days. INGAP Peptide treatment resulted in a significant increase in the number of islets compared with placebo-treated animals (FIG. 2). The islet neogenesis effect was manifested by production of more insulin and an increase in the number of islets in the pancreata. Newly formed β-cells appeared in the wall of, and budding from, pancreatic ducts. These insulin-positive cells resulted from ductal epithelial cell differentiation and islet cell growth, and their appearance was proportional to the dose and duration of treatment with INGAP Peptide. Over longer periods of treatment, these cells migrated away from the duct and formed islets in the parenchyma of the pancreas. After 10 consecutive days of INGAP Peptide administration, there was a 30% increase in islet number, and by 30 days there was a doubling of the number of islets in the tissues, consistent with the prior observations using ilotropin, INGAP, and cellophane wrapping in animal models.

### EXAMPLE 3 IN VIVO EFFICACY STUDY

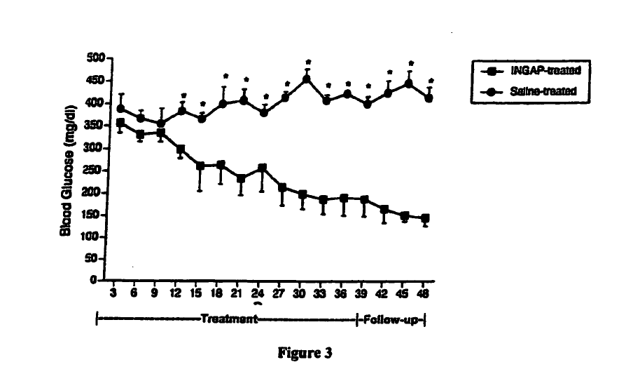

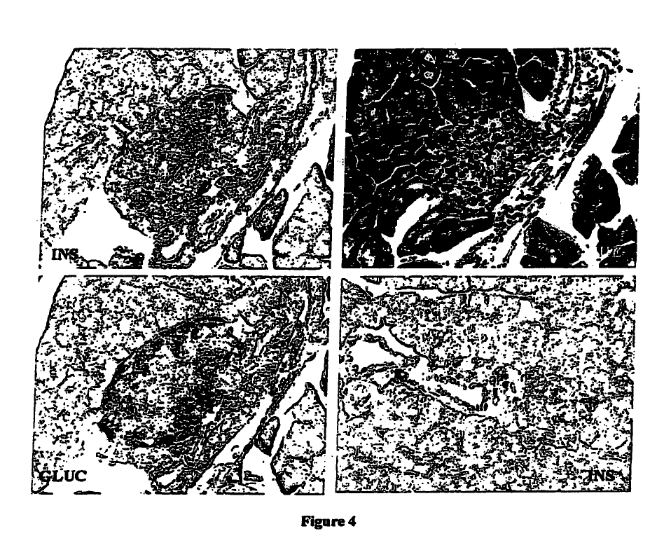

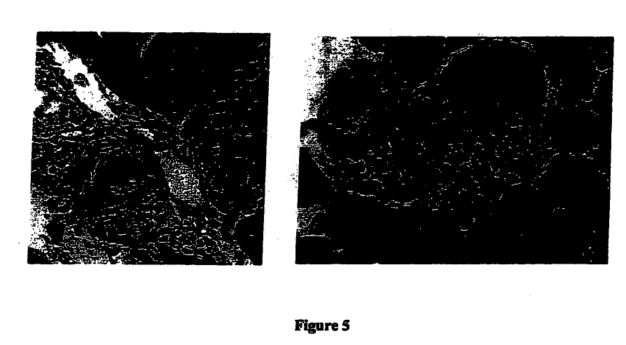

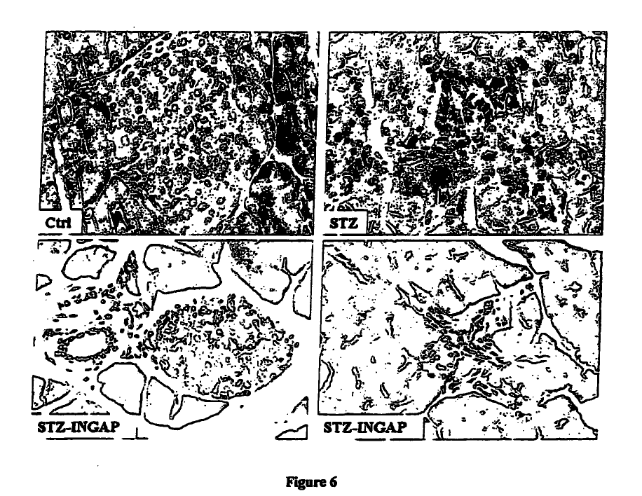

C57BL/6J mice were made diabetic with STZ (35 mg/kg/day×5 days) and divided in INGAP Peptide-treated (250 [symbol] g twice daily) and saline control groups of 4 animals each. All four of the INGAP Peptide-treated animals had their blood glucose concentrations restored to normal, whereas all of the saline-treated mice remained hyperglycemic (FIG. 3). After 39 days, dosing was stopped and further observation showed durability of the effect to 48 days, when the study was terminated. Histopathologic evaluation of INGAP Peptide-treated animals showed both the presence of normal-appearing islets and areas of new islet formation, including a normal complement and distribution of insulin and glucagon secreting cells (FIGS. 4, and 6). The appearance of glucagon producing cells is noteworthy since glucagon plays a major role in the defense against hypoglycemia. This feature of the INGAP Peptide induced islet neogenesis could help to reverse the impaired counter regulatory control of hypoglycemia associated with the overzealous treatment of diabetes. Hypoglycemia was not observed in any of the INGAP Peptide-treated animals. In saline-treated control animals, no new islet formation was observed. INGAP Peptide administration induced transdifferentiation of ductal cells as evidenced by cells expressing the transcription factor PDX-1 (FIG. 5). Islets in the saline-treated STZ-diabetic animals showed heavy inflammatory cell infiltrate and were necrotic. In INGAP Peptide-treated animals, inflammation was markedly reduced and the islets appeared healthy (FIG. 6).

FIG. 4 shows the immunocytochemical characteristics of the pancreas of streptozotocin-treated C57BL/6J mice further treated with INGAP Peptide. The upper left panel shows an islet still associated with a segment of duct epithelium stained with anti-insulin antibody, which demonstrates a normal presence and distribution of insulin protein. The lower left panel shows the same islet stained with a mixture of anti-glucagon and anti-somatostatin antibodies also demonstrating a normal distribution of these islet call proteins in the islet mantle region. The upper right panel shows a newly formed islet budding off a duct stained with H & E stain. The lower right panel shows an insulin-positive cell in the wall of the duct.

### EXAMPLE 4 31-DAY MOUSE STUDY (REPEAT DOSING)

A repeat-dose toxicology study was conducted in mice with 31 days of daily injection of INGAP Peptide at 0, 2, 20, and 100 mg/kg/day. In this study, four treatment groups of 10 males and 10 females each were allocated, as were two groups of recovery animals (5 males and 5 females). Blood was collected at termination and necropsies were performed for gross and microscopic observations. Clinical pathology and serum levels were evaluated in approximately half the animals in each group. Selected organs (brain, adrenal, heart, kidney, liver, lung, pancreas, and spleen) were weighed and relative organ weights were calculated. A section of the pancreas was removed and frozen in liquid nitrogen for evaluation for insulin content and sections of pancreas tissue were submitted for independent microscopic examination. Recovery animals were terminated 28 days after cessation of dosing. Various parameters for further study as well as potentially drug-related abnormal findings were evaluated to determine the reproducibility and potential clinical significance.

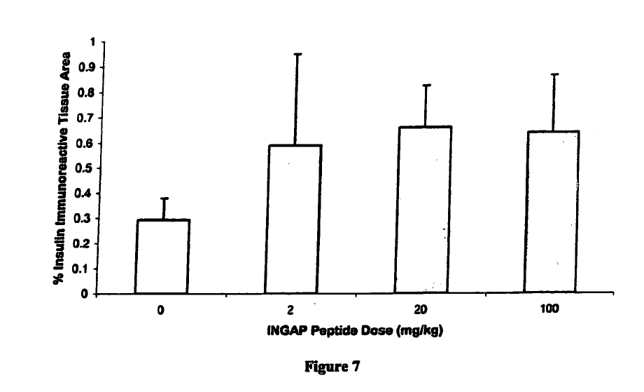

Administration of INGAP Peptide by IM injection for 31 consecutive days produced no dose-related adverse effects when evaluated as cessation of dosing and through 28 days post-treatment. Injection site irritation was observed in males and females with increased frequency at the highest dose, but was no longer observed in recovery animals at that same dose, showing reversibility of irritation. Extramedullary hematopoiesis in the spleen was seen in one male animal at the high dose in this 31-day study. No microscopic evidence of inflammatory cell infiltration, edema, necrosis or atrophy was observed. The salient observation was the increase in the number of small islets, both duct-associated, and in amongst the acinar tissue. Serum levels of INGAP Peptide at 2 hours after dosing for 31 consecutive days were below the limits of quantitation. Pharmacological activity as measured by an increase in insulin-positive tissue area was observed in these animals (FIG. 7). The results suggest that the no adverse effect level (NOAEL) greater than 100 mg/kg in CD-1 mice with 31-day dosing.

### EXAMPLE 5 34-DAY DOG STUDY (REPEAT DOSING)

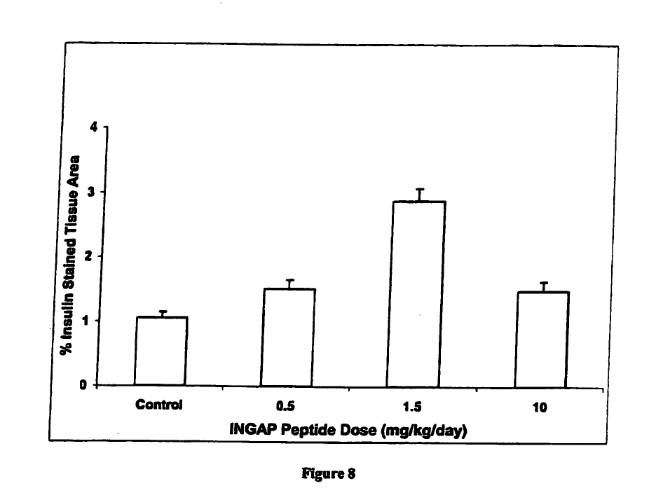

A repeat-dose toxicology study was conducted in beagle dogs for 34 days with daily IM injection of INGAP Peptide at 0, 0.5, 1.5, and 10 mg/kg/day. Pancreatic tissue was obtained for quantitation of β-cell mass by immunohistochemistry from animals sacrificed on Day 34, at termination of treatment, and from recovery animals sacrificed 25 days after termination of treatment. Pancreatic β-cell mass was increased following INGAP Peptide administration (see FIG. 8). These results indicate that IM injections of INGAP Peptide in the range of doses studied achieve a biologically important response in the normal beagle dog. Furthermore, an in-depth review of the pancreatic tissue sections showed no changes such as edema, inflammatory cell infiltration, necrosis or atrophy.

### EXAMPLE 6 HUMAN CLINICAL STUDIES

Doses are often based on the results of efficacy and safety studies in animals. Two doses of INGAP Peptide, 7.5 mg (0.125 mg/kg, or 4.625 mg/m² for a 60 kg patient) and 120 mg (1.6 mg/kg, or 74 mg/m² for a 60 kg patient) are exemplified in the treatment of type I or type II diabetes mellitus. The following parameters are evaluated to determine efficacy of INGAP Peptide treatment.
1. A reduction of fasting glucose levels by >35 mg/dl while the total insulin dose is maintained.
2. A reduction of insulin dose by 25% with fasting glucose levels maintained in the normal range as determined by the American Diabetes Association (ADA) criteria.
3. An increase in fasting C-peptide >1 ng/ml is obtained. An increase in C-peptide of >2 ng/ml in response to Sustacal® (Boost®) is obtained.

Each patient is randomized to receive one single intramuscular injection of INGAP Peptide. After evaluating efficacy and safety data, patients could be given further INGAP Peptide injections as deemed appropriate by the physician.

The following table summarizes a partial list of assessments that are made on patients receiving the INGAP Peptide or placebo treatment.

TABLE 2
Schedule of Assessments

| | | | Treatment Period | | | | | | Follow-Up | | | |
|---|---|---|---|---|---|---|---|---|---|---|---|---|
| Procedure | | | Days 1-34 | | | | | | Days 35-63± | | | |
| Visit Day | Screen | Baseline | 1 | 7 | 14 | 21 | 28 | 34 | 42 | 49 | 56 | 63 |
| Physical Examination | X | X | | | | | | X | | | | X |
| Vital Signs | X | X | X | X | X | X | X | X | X | X | X | X |
| Clinical laboratory | X | X | | X | X | X | X | X | | | | X |
| tests | | | | | | | | | | | | |
| Plasma PK for | | X | X | X | X | X | X | X | X | | | |
| INGAP Peptide | | | | | | | | | | | | |

### Stimulated C-Peptide

Stimulated C-peptide tests are performed in the morning after an overnight fasting period of at least 10 hours. The tests are performed only if the fasting glucose is between 80 and 250 mg/dl. Patients can take their diabetes medications the evening before, but should not take them the morning of the test until the test is completed. Blood samples for the determination of C-peptide are drawn immediately before Boost® ingestion, and at 0.5, 2, and 4 hours post-ingestion. Boost® is administered through ingestion. Patients are considered insulin deficient if their fasting C-peptide is <1.0 ng/ml and their maximum stimulated C-peptide value is <2.0 ng/ml.

As a result of the treatment, patients receiving the INGAP Peptide show improved sugar tolerance, a reduction in fasting glucose level, a reduction in insulin does required, an increase in fasting C-peptide level, and an increase in C-peptide level in response to Boost®. Patients receiving placebo treatment show no such improvements.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A method of regenerating functional alpha or beta cells in the islets of Langerhans of a non-rodent mammal comprising administration of an effective amount of a peptide comprising Seq ID No. 3.

2. The method of claim 1 wherein the normal biofeedback mechanisms for insulin regulation are maintained.

3. The method according to claim 2 wherein the pharmaceutical composition comprises from about 0.1 mg to about 300 mg of the polypeptide.

4. The method according to claim 2 wherein the pharmaceutical composition comprises from about 5 mg to about 150 mg of the polypeptide.

5. The method of claim 2 wherein the administration is at a frequency selected from the group consisting of daily, twice daily, three times daily, weekly, biweekly, monthly, continuous infusion and combinations thereof.

6. The method of claim 2 wherein the pharmaceutical composition is administered for a period of at least 30 days.

7. A method of increasing beta cell mass in the islets of Langerhans of a non-rodent mammal comprising administration of an effective amount of a peptide comprising Seq ID No. 3.

8. The method of claim 6 wherein the normal biofeedback mechanisms for insulin regulation are maintained.

9. The method according to claim 6 wherein the pharmaceutical composition comprises from about 0.1 mg to about 300 mg of the polypeptide.

10. The method according to claim 6 wherein the pharmaceutical composition comprises from about 5 mg to about 150 mg of the polypeptide.

11. The method of claim 6 wherein the administration is at a frequency selected from the group consisting of daily, twice daily, three times daily, weekly, biweekly, monthly, continuous infusion and combinations thereof.

12. The method of claim 6 wherein the pharmaceutical composition is administered for a period of at least 30 days.

13. A method of establishing normal physiological glucose regulation in a mammal in need of such a treatment comprising administering to the mammal a therapeutically effective amount of a peptide comprising Seq ID No. 3.

14. The method according to claim 13 wherein the pharmaceutical composition comprises from about 0.1 mg to about 300 mg of the polypeptide.

15. The method according to claim 13 wherein the pharmaceutical composition comprises from about 5 mg to about 150 mg of the polypeptide.

16. The method of claim 13 wherein the administration is at a frequency selected from the group consisting of daily, twice daily, three times daily, weekly, biweekly, monthly, continuous infusion and combinations thereof.

17. The method of claim 13 wherein the pharmaceutical composition is administered for a period of at least 30 days.
